(19) United States
(12) Plant Patent Application Publication
(10) Pub. No.: US 2008/0005818 P1
(43) Pub. Date: Jan. 3, 2008

(54) Hibiscus plant named 'Jodi'

(76) Inventor: David Smith, Powell, OH (US)

Correspondence Address: GALLAGHER & DAWSEY CO., L.P.A., P.O. BOX 785, COLUMBUS, OH, 43216, US

(21) Appl. No.: 11/480,005

(22) Filed: Jun. 30, 2006

(51) Int. Cl. A01H 5/00

(52) U.S. Cl. PLT/257

(57) ABSTRACT

A new cultivar of Hibiscus, Hibiscus syriacus ‘Jodi,’ characterized by three color irregular variegation (Dark green, light green, and golden yellow); a large, pure white flower; a longer blooming duration; two additional seasons of ornamental qualities because of variegated leaves; and triploid (sterile) genetic character.

### CROSS-REFERENCE TO RELATED APPLICATIONS

None

### FEDERAL SPONSORSHIP

No federal research and development funds were used in the development of this plant.

### LATIN NAME OF THE GENUS AND SPECIES OF THE PLANT CLAIMED

### BOTANICAL CLASSIFICATION

Hibiscus syriacus

### VARIETAL DENONMINATION

‘Jodi’

### BACKGROUND OF THE INVENTION

Hibiscus syriacus ‘Jodi’ is a new cultivar of the common Rose-of-Sharon. The plant will be useful as an improved ornamental Hibiscus. The plant has irregular variegation of yellow and green which provides enhanced spring and summer ornamental characteristics. The plant also has sterile, single, white flowers which in late summer provide a longer duration of flowering than other Hibiscus. Hibiscus syriacus ‘Jodi’ is hardy from Zone 5 to Zone 8. The plant can be used as a specimen, in borders, as a hedge, or as a small tree.

Hibiscus syriacus ‘Jodi’ is a spontaneous, genetic mutation from Hibiscus syriacus ‘Diana’. The discovery was made at Smiths Gardens; a wholesale nursery located in Worthington, Ohio. The mutation has been propagated asexually by softwood cuttings and observed in numerous controlled environments to test for stability, hardiness, vigor, and disease and insect resistance. Clones or propagules of the claimed plant are identical to the original plant in all distinguishing characters, demonstrating that the claimed plant is stable.

### SUMMARY OF INVENTION

Novel Characteristics—as compared to ‘Diana’

1. Three color irregular variegation (Dark green, light green, and golden yellow)

Novel Characteristics—as compared to all other Hibiscus

1. Triploid (sterile)

2. Large, pure white, single flower

3. Longer blooming duration

4. Two additional seasons of ornamental qualities because of variegated leaves.

### BRIEF DESCRIPTION OF THE PHOTOGRAPHS

Without limiting the scope of the present invention as claimed below and referring now to the drawings and figures:

FIG. 1 is a close up view of the leaves of ‘Jodi.’ The colors in the photographs are as close as possible with the photographic and printing technology utilized. The color values cited in the detailed botanical description accurately describe the color of the new Hibiscus.

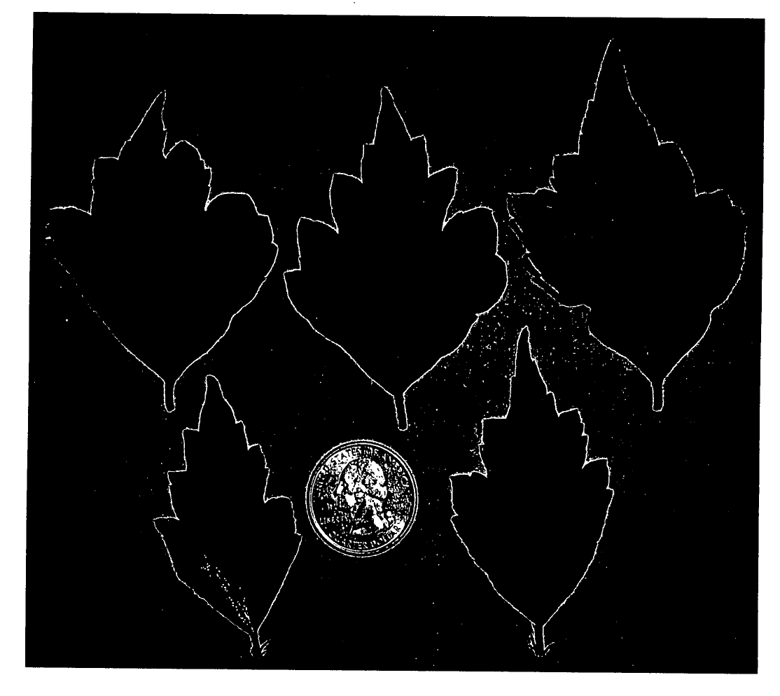

### DETAILED BOTANICAL DESCRIPTION OF THE PLANT

The following is a detailed description of the new cultivar grown outdoors derived from evaluation rooted softwood cuttings grown in one gallon containers, two and three year-old specimens grown in 3 gallon containers, and multiple field grown specimens grown under different climactic conditions. All specimens were grown in Powell, Ohio, United States of America, under natural light conditions. The specimens have performed well in Zone 5b (−10 to −15 degrees F.) of the USDA Plant Hardiness Zone Map Misc. Pub. 1475 Henry Cathey January 1990. The new Hibiscus has been observed to do well in daytime temperatures up to 100 degrees F. The phenotype of the new cultivar may vary with variations in environmental, climatic, and cultural conditions, as it has not been tested under all possible environmental conditions. The color determination is in accordance with the 2001 R.H.S. Color Chart of The Royal Horticultural Society, London, England, except where general color terms of ordinary dictionary significance are used.
Botanical classification: ‘Jodi’ is a cultivar of Hibiscus syriacus.
Parentage: Hibiscus syriacus ‘Jodi’ is a cultivated genetic sport of Hibiscus syriacus ‘Diana’.
Growth habit of the plant described as to the shape of the plant at maturity, and branching habit: ‘Jodi’ is a shrub or small tree with upright branching habit. At maturity, the plant has an upright oval shape.
Characteristics of the plant in winter dormancy: The winter habit is one of medium texture. The outline is a semi-broad upright oval shape.
Complete botanical description of bark, buds, blossoms, leaves, and fruit:
   Bark.—older bark is grey with semi-smooth texture. Younger stems have maroon color and are very smooth.
   Buds.—are not significant and are located by the leaf scars of the previous year.
   Blossoms.—Flowers are generated on current year's growth. They are five petaled, perfect, solitary, single, pure white, 3″ to 5″ across and occur in August through September. The flowers on this cultivar start a week later than other varieties but lasts much longer into the season. The flowers also remain open at night. The flowers of the parent plant are thought to be sterile and observation of Hibiscus syriacus ‘Jodi’ has generated no apparent self-seeding.
   Leaves.—The leaves are alternately located on the stem. The leaves have three lobes with palmate veination. The leaf shape is ovate with a cuneate base with acute apices. The margins are fixed with rounded serrations. The leaves are 2 to 4 inches in length and 1¾ to 2½ inches in width at the widest point. The upper surface of the leaf has a waxy texture. The lower leaf surface is dull with minor pubescence along the veination. The petiole is 1/4″ to 1″ in length. The colors are best described using Royal Horticultural Society Color Charts. The leaves have three colors. The darkest green is the same shade as RHS Fan3 137A. The light green is best described as RHS Fan3 138B. The yellow is best described as RHS Fan3 153C. The color pattern is irregular leaf by leaf. The variegation follows the veination in irregular unpredictable patterns.
   Fruit.—the fruit is dehiscent five-valved capsule. The fruit starts out a light green about ¾″ long. After the capsule opens, it turns brown and persists through winter.
Plant characteristics: This Hibiscus has a medium growth rate. The ultimate height is 8′-12′ in height and 6′-10′ wide. The plant is hardy from zone 5 to zone 8. This particular cultivar has shown little if any self-seeding, which is a nuisance with many of the other cultivars. The flowers show no fragrance. This cultivar has shown no significant susceptibility to diseases or insects. The plant roots easily from softwood cuttings taken in June to July when treated with 1000 ppm IBA. I claim:

1. A new and distinct cultivar of Hibiscus plant named ‘Jodi’ as herein illustrated and described.
